(19) United States
(12) Patent Application Publication
(10) Pub. No.: US 2007/0189990 A1
(43) Pub. Date: Aug. 16, 2007

(54) Ultraviolet absorber for cosmetics and polymeric materials

(76) Inventors: Anthony East, Madison, NJ (US); Yi Zhang, Harrison, NJ (US); Michael Jaffe, Maplewood, NJ (US)

Correspondence Address: Davis, Brown, Koehn, Shors & Roberts, P.C.;Suite 2500, The Financial Center, 666 Walnut Street, Des Moines, IA, 50309-3993, US

(21) Appl. No.: 11/355,936

(22) Filed: Feb. 16, 2006

(51) Int. Cl. A61K 8/49; C07D 307/02

(52) U.S. Cl. 424/59; 549/478

(57) ABSTRACT

Esters of cinnamic acids with sugar alcohols and anhydro-sugar alcohols are described. The esters have ultraviolet light absorbing properties. In particular, the diester of ferulic acid with isosorbide is a UV-absorber derived from natural materials and has use as a UV-absorber for polymeric materials and as a UV-absorber for cosmetics.

The invention was made with Government support under Contract DE-AC0676RLO 1830, awarded by the U.S. Department of Energy. The Government may have certain rights in the invention.

### BACKGROUND OF THE INVENTION

The invention relates to absorbers of ultraviolet light as stabilizers for cosmetics and polymeric materials and, more specifically, to the esters of cinnamic acids with biomass derived polyols and their derivatives.

It is well known that ultraviolet light causes certain polymeric materials to discolor, become brittle, crack, and otherwise degrade, especially in the presence of atmospheric oxygen. A variety of materials have been used to ameliorate the effects of UV radiation, including UV-absorbers, and certain anti-oxidants such as hindered phenols, nickel chelates, aryl esters, and certain hindered amines. Ultraviolet stabilizers protect light-sensitive materials from degradation by ultraviolet light. The stabilizers are typically colorless or almost colorless organic substances.

Ultraviolet radiation initiates degradation in light sensitive materials including polymers because the amount of energy in photons of ultraviolet radiation may be above the dissociation energy of the covalent bonds in these materials. The shorter the wavelength, the more damaging is the UV radiation. Even the solar radiation wavelengths normally present at the Earth's surface after filtering through the atmosphere from the sun can cause such damage by cleaving bonds to form free radicals. These radicals react through a number of different pathways to produce a variety of substances inimical to the materials it is desired to protect.

There are many low molecular weight UV-absorbers based on various chromophores such as benzotriazoles, 2-hydroxybenzophenones and cinnamates. Many of these chromophores can be functionalized with reactive groups such as hydroxyl, amino or, particularly, carboxyl groups, so that they are capable of reacting with other molecules such as polyhydroxy compounds to form large molecular weight species. These are more permanent and less fugitive additives, particularly important when admixed with polymers. Cinnamic acids are a particularly useful class of UV-stabilizers because they have the desired UV-VIS absorption spectra and being carboxylic acids can form esters with monohydric or polyhydric alcohols. Several hydroxy-cinnamic acids occur naturally in higher plants including sinapic acid, caffeic acid, hesperetic acid and, the most useful, ferulic acid (3-methoxy-4-hydroxy-cinnamic acid) which occurs widely in rice hulls. Esters of this acid have been used as UV stabilizers as described in U.S. Pat. No. 5,908,615.

Biomass derived compounds such as sugar alcohols and their dehydrated products are known useful polyols. In particular, those bicyclic ether derivatives known as the bisanhydro-hexitols, made by doubly dehydrating hexitols such as sorbitol and mannitol to give isosorbide or isomannide may be reacted with cinnamic acids. The diester of ferulic acid with isosorbide seems a promising candidate for a UV-absorber, one that is derived from two moieties that are known to be biodegradable. Such as UV-absorber from a naturally occurring cinnamic acid and a biomass-derived anhydrosugar will have obvious desirability as a UV-absorber for personal care products such as cosmetics.

### SUMMARY OF THE INVENTION

The invention consists of ultraviolet light absorbing compounds that are esters of a cinnamic acid, preferably ferulic acid, with either a polyhydric sugar alcohol or an anhyro-derivative of polyhydric sugar alcohols preferably isosorbide. The UV-absorbing compounds are useful as UV-absorbers or UV-stabilizers for addition to polymeric materials to prevent degradation of the polymer by ultraviolet light. The UV-absorbing compounds are also useful as additions to cosmetic compositions for use as sunscreens.

In preferred embodiments, the cinnamic acid is selected from the group of plant-derived cinnamic acids, including ferulic acid, sinapic acid, 4-hydroxycinnamic acid and caffeic acid. Preferred polyhydric sugar alcohols include sorbitol and mannitol and preferred anhydro-derivatives of the polyhydric sugar alcohols include isosorbide and isomannide.

The ultraviolet light absorbing compounds of the present invention can be derived from materials of natural origin and are found to have strong absorbance peaks in the UVA and UVB ranges, making the compounds particularly suited for use in the cosmetic industry.

### BRIEF DESCRIPTION OF THE DRAWINGS

FIG. 1 is an ultraviolet light absorbance spectrum of isosorbide bis ferulate made according to the present invention.

### DETAILED DESCRIPTION OF PREFERRED EMBODIMENTS

This invention includes UV absorbers derived from the reaction of esters of a cinnamic acid with a polyhydric sugar alcohol or an anhydro-derivative of polyhydric sugar alcohols.

Cinnamic acids are 3-arylacrylic acids of the general formula:

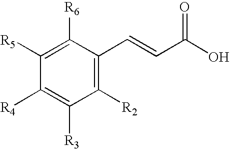

Cinnamic acids are widely distributed in nature and can be easily synthesized by condensation of aromatic aldehydes (ArCHO) with activated methylene groups in molecules such as malonic acid, malonic esters or cyanoacetic esters. Cinnamic acids, and their simple alkyl esters, particularly those with electron-rich constituents on the aromatic ring, such as hydroxy, methoxy and other alkoxy groups, and especially when the said substituents are in the 2-position or 4-position on the aromatic ring, have chromophores that strongly absorb light in the ultraviolet (UV) range and as such are used as components of UV-absorber materials. Examples of esters of cinnamic acids suitable for use in this invention include but are not limited to isosorbide 2,5-bis ferulate, isomannide bis ferulate, isoidide bis ferulate, isosorbide 2-monoferulate, isosorbide 5-monoferulate and isoidide monoferulate and mixed diesters of diols with cinnamic acids and other aliphatic or aromatic acids such as isosorbide 2-acetate-5-monoferulate, and other analogous compounds. Derivatives such as the polycinnamates of sorbitan and mannitan, sorbitol and mannitol are included.

Polyhydric sugar alcohols are defined as those polyols derived by the reduction (or hydrogenation) of the nominal aldehyde group in simple sugars, notably tetroses, pentoses and hexoses. Polyhydric sugar alcohols are most commonly derived from biomass sources, such as corn, natural starches and degradable cellulose-derived carbohydrate materials. Examples of polyhydric sugar alcohols suitable for use in this invention include but are not limited to sorbitol, mannitol, iditol, xylitol, erythritol, threitol, and the like. Anhydro-derivatives of the polyhydric sugar alcohols may also be used in practicing this invention. Anhydro-derivatives of the polyhydric sugar alcohols are produced by internal dehydration of various hydroxyl-groups, thus generating cyclic ether rings. Anhydro-derivatives suitable for this invention include single ether ring monoanhydrohexitols such as sorbitan and mannitan and the bisanhydrohexitols (which have two cyclic rings) isosorbide, isomannide and isoidide. Isosorbide, isomannide and isoidide have the characteristics of bis secondary alcohols attached to two cis-fused tetrahydrofuran rings and as such possess the properties of a diol and an ether. They are very soluble in water and biologically harmless. Isomannide and isoidide are both symmetrical molecules insofar as the two hydroxyls are chemically and structurally equivalent, both endo in the case of isomannide and both exo in the case of isoidide. However, isosorbide has one endo and one exo group, so that mono-acylation gives rise to two different non-equivalent ester products, namely a 2-ester or a 5-ester. This does not affect the usefulness of isosorbide cinnamates as UV absorbers but can complicate the isolation of pure products in the case of isosorbide.

Ferulic acid is 3-methoxy-4-hydrocinnamic acid and is a natural product occurring widely in higher plants. Its structure 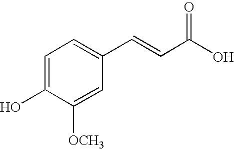
has a chromophore that absorbs UV light in the correct range to be useful as a sunscreen and anti-degradant in cosmetics and polymeric materials. Ferulic acid also has valuable anti-inflammatory, antibacterial, and antipruritic properties (U.S. Pat. No. 6,114,377). Unfortunately, the free acid tends to decompose on the skin under aqueous conditions and its administration is difficult (U.S. Pat. No. 6,632,444). A recent patent discloses long chain fatty alcohol monoesters of ferulic acid as more stable compositions for cosmetic use (U.S. Pat. No. 5,908,615).

Ferulic acid can be synthesized from vanillin by the method of Pearl (Pearl and Bayer, J Org Chem., 1951, pp. 219). In the Pearl synthesis (a modification of the earlier Vorsatz Reaction) vanillin is reacted with malonic acid in pyridine and a trace of a secondary amine such as piperidine at room temperature for one month. In an improved process, the reaction mixture is stirred at a controlled 40° C. and a bubbler tube fitted to the reaction vessel to monitor the evolution of CO₂. When the production of bubbles ceases, the reaction is over. The improved method gave 80% yields of high purity ferulic acid in a shorter time of two weeks. Ferulic acid can also be obtained by the condensation of vanillin with malonic esters in the presence of a piperidine catalyst to give vanillylidene-malonate esters. Such methods are described in U.S. Pat. No. 5,175,340 and a further improvement is given in Chinese Patent 1,544,409. Other such compounds and their syntheses are described in US Patent Application 2004/0247536. Vanillylidene-malonate esters can be degraded to ferulic acid by saponification with aqueous alkali and spontaneous decarboxylation of the product after acidification. Yet a further method is achieved by the condensation of vanillin with a cyanoacetate ester to give an alpha-cyanocinnamate and its subsequent hydrolysis and decarboxylation (see US Pat. Appl. 2004/0247536).

In order to react ferulic acid or other hydroxy-cinnamic acids with a polyol, a method must be used which is selective for the reaction of the carboxy group with the polyol without any self-condensation with the phenolic hydroxy-group, which would complicate the process. One way that has been found to work well is to use dicyclohexyl carbodi-imide as a dehydrating agent in the presence of a catalyst such as 4-dimethyl-aminopyridine. A similar method has been described in U.S. Pat. No. 6,441,217 to synthesize phenolic esters of ferulic acid. The reaction takes place at moderate temperatures (20-50° C.) in tetrahydrofuran as a reaction medium, the insoluble by-product dicyclohexylurea precipitating readily from the reaction mixture.

Another route protects the 4-hydroxy group of ferulic acid by acetylation of the phenolic hydroxyl with acetic anhydride to give acetylferulic acid, which is reacted with oxalyl chloride to make the acid chloride. This is not isolated as such but reacted directly with a polyol (such as isosorbide) in the presence of pyridine or triethylamine as an acid acceptor at low temperatures (for example −10 to 25° C.) with or without the presence of an inert solvent. The product is the O-acetyl derivative of the desired ferulate ester. However, the protective phenolic acetate group is easily removed by stirring the product at room temperature in methanol containing a little methanesufonic acid, which removes the acetate group by methanolysis without affecting the desired aliphatic ester groups.

Yet a third route is exemplified by reacting isosorbide (or another polyol) at an elevated temperature with an excess of ethyl cyanoacetate in the presence of an ester exchange catalyst, such as a titanium, zinc or tin derivative, and distilling away the liberated ethanol so as to form isosorbide biscyanoacetate. Cyano-esters are particularly reactive in condensation reactions and will react with vanillin and a piperidine catalyst to generate in situ the alpha-cyanoferulate bis ester, which is a strong UV absorber.

As well as ferulic acid, other hydroxycinammic acids such as sinapic acid and those mentioned earlier may be similarly employed.

Synthetic procedures are set out in the Examples below.

### EXAMPLE 1

### Preparation of Ferulic Acid

In a 3000 mL 3-neck flask, fitted with a stir-bar, long stem thermometer and a bubbler tube, were placed 99% pure vanillin (100.0 g; 0.66 mole) crystalline malonic acid, (140.0 g; 1.34 mole), anhydrous pyridine (500 mL) and 4-methylpiperidine (2.0 mL). The system was stoppered and warmed on a heating mantle to a controlled 40° C. and stirred magnetically. As soon as the methylpiperidine was added, the colorless solution went yellow. Within one day a steady stream of bubbles emerged from the bubbler tube as carbon dioxide was evolved. The reaction was left to stir and after 20 days the evolution of gas had ceased. The warn solution was made strongly acid by adding a mixture of 200 mL concentrated hydrochloric acid and cracked ice. Nothing precipitated at first, but after standing several hours in a refrigerator at 0° C., a mass of colorless needles was deposited. These were filtered off and washed with a little water and the filtrate concentrated on the rotary evaporator to yield additional product. The combined fractions were recrystallized from 50/50 ethanol/water and filtered hot to yield long colorless needles of ferulic acid, (99 g; 77% theoretical) with a melting point of 172-4° C. (theoretical 171° C.-174° C., from the Chemical Rubber Handbook and the Merck Index, respectively).

### EXAMPLE 2

### Preparation of Isosorbide bis Ferulate (0.1 mole isosorbide scale)

A 2000 mL four-neck flask was fitted with an over-head paddle stirrer, a long stem thermometer, a gas inlet tube and a short air condenser topped with a bubbler tube. All glassware was thoroughly dry. The flask was charged with 500 mL dry tetrahydrofuran (THF), dried isosorbide (14.6 g, 0.10 mole), recrystallized ferulic acid (48.0 g, 0.25 mole) and 4-dimethylaminopyridine, DMAP, (1.0 g). The mixture was stirred at room temperature under a slow steam of nitrogen gas until a clear colorless solution formed. Meanwhile, 50 g (0.24 mole) of dicyclohexylcarbodiimide was heated at 45° C. in an oven until it melted and the liquid added all at once to the flask and stirred. The mixture at once went bright yellow and the temperature rose to about 40° C., then slowly fell again. It soon went cloudy as the byproduct, dicyclohexylurea, began to precipitate. After stirring for five hours, the mixture was left to stand overnight under nitrogen. Next day the urea was filtered off and washed with THF until the solid on the filter was colorless and the yellow filtrate evaporated to dryness to leave a yellow gummy solid. The solid was dissolved in dichloromethane and washed twice with dilute acetic acid and once with water to remove the DMAP, dried over anhydrous sodium sulfate and evaporated down again to give a yellow solid weighing 46.4 g (93% theory).

### EXAMPLE 3

### UV-absorbance Spectrum of Isosorbide bis Ferulate

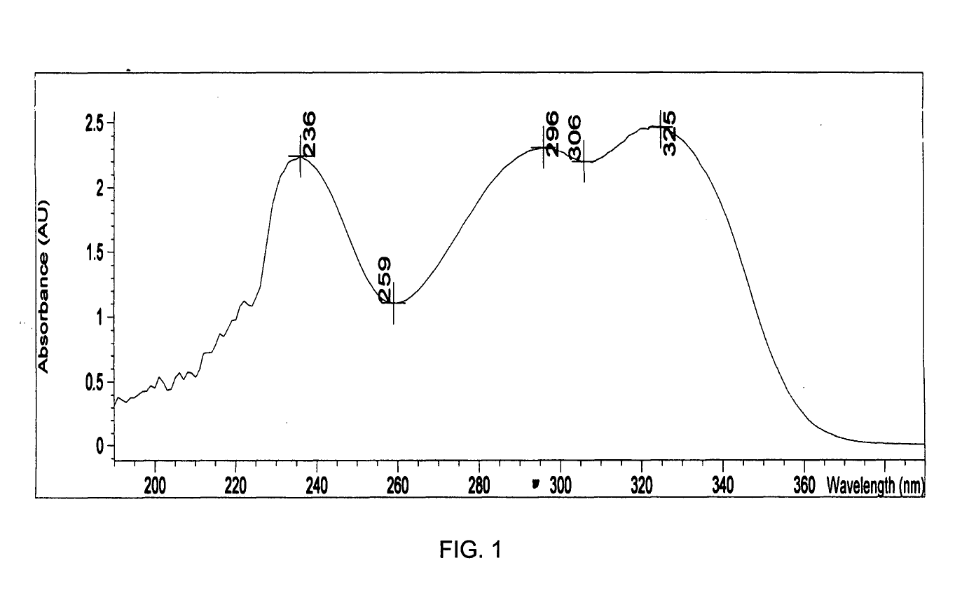

The UV spectrum of the compound from Example 2 was measured using a single beam HP 8453 UV-Visible spectrophotometer. The solvent was dichloromethane, the cuvettes had a 1.0 cm path length and the concentration was 0.0063% w/v. The spectrum is illustrated in FIG. 1. The main absorption peaks were at 236 nm (ε=17720), 296 nm, (ε=18220) and 325 nm (ε=19450).

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

What is claimed is:

1. An ultraviolet light absorbing compound, comprising esters of a cinnamic acid with a polyhydric sugar alcohol or an anhydro-derivatives of polyhydric sugar alcohols.

2. A compound as described in claim 1, wherein the cinnamic acid is selected from the group of plant-derived cinnamic acids.

3. A compound as defined in claim 2, wherein the plant-derived cinnamic acid is selected from the group consisting of ferulic acid, sinapic acid, 4-hydroxycinnamic acid, and caffeic acid.

4. A compound as defined in claim 1, wherein the polyhydric alcohol is selected from the group consisting of sorbitol and mannitol.

5. A compound as defined in claim 1, wherein the polyhydric alcohols are derived from plant sources.

6. A compound as defined in claim 1, wherein the anhydro-derivatives are selected from the group consisting of isosorbide and isomannide.

7. A formulation, comprising the compound as defined in claim 1 incorporated into a polymeric material as a stabilizer to prevent degradation of the polymeric material by ultraviolet light.

8. A sunscreen formulation, comprising a cosmetic incorporating a compound as defined in claim 1.

9. An ultraviolet light absorbing compound, comprising esters of a cinnamic acid with a compound selected from the group consisting of polyhydric sugar alcohols and anhydro-derivatives of polyhydric sugar alcohols.
